(19) United States
(12) Patent Application Publication
(10) Pub. No.: US 2008/0188927 A1
(43) Pub. Date: Aug. 7, 2008

(54) ENDOVASCULAR IMPLANT WITH ACTIVE COATING

(75) Inventors: Roland Rohde, Burgdorf (DE); Katrin Sternberg, Rostock (DE); Tobias Diener, Erlangen (DE)

Correspondence Address: HAHN LOESER & PARKS, LLP, One GOJO Plaza, Suite 300, AKRON, OH, 44311-1076, US

(73) Assignee: BIOTRONIK MESS- UND THERAPIEGERAETE GMBH & CO., Berlin (DE)

(21) Appl. No.: 11/951,620

(22) Filed: Dec. 6, 2007

Related U.S. Application Data
(62) Division of application No. 10/639,246, filed on Aug. 11, 2003, now abandoned.

(30) Foreign Application Priority Data
Aug. 13, 2002 (DE) 102 37 571.2

(51) Int. Cl. A61F 2/82

(52) U.S. Cl. 623/1.42

(57) ABSTRACT

The invention concerns an endovascular implant, in particular a stent, with an at least portion-wise active coating. The object of the present invention is to provide locally therapeutic formulations for the treatment of stenosis or restenosis. The implants modified in accordance with the invention are to ensure improved compatibility, in particular in regard to any inflammatory and proliferative processes in the tissue environment. That is achieved in that the active coating includes, as an active substance: 1) PPARα-agonists, PPARδ-agonists or a combination thereof; 2) an RXR-agonist; or 3) a combination of PPAR-agonists and RXR-agonists.

### CROSS REFERENCE TO RELATED APPLICATIONS

This application is a divisional application of co-pending U.S. patent application Ser. No. 10/639,246, filed Aug. 11, 2003.

### FIELD OF INVENTION

The invention concerns an endovascular implant, in particular a stent, comprising at least one portion-wise active coating and the use of PPAR-agonists and RXR-agonists for the local treatment of stenosis or restenosis.

### BACKGROUND OF THE ART

One of the most frequent causes of death in Western Europe and North America is coronary heart diseases. According to recent knowledge, in particular inflammatory processes are the driving force behind arteriosclerosis. The process is supposedly initiated by the increased deposit of low-density lipoproteins (LDL-particles or β-lipoproteins) in the intima of the vessel wall. After penetrating into the intima the LDL-particles are chemically modified by oxidants. The modified LDL-particles in turn cause the endothelium cells which line the inner vessel walls to activate the immune system. As a consequence monocytes pass into the intima and mature to macrophages. In conjunction with the T-cells which also enter inflammation mediators such as immune messenger substances and proliferatively acting substances are liberated and the macrophages begin to receive the modified LDL-particles. The lipid lesions which are formed from T-cells and the macrophages which are filled with LDL-particles and which by virtue of their appearance are referred to as foam cells represent an early form of arteriosclerotic plaque. The inflammation reaction in the intima, by virtue of corresponding inflammation mediators, causes smooth muscle cells of the further outwardly disposed media of the vessel wall to migrate to under the endothelium cells. There they replicate and form a fibrous cover layer from the fiber protein collagen, which delimits the subjacent lipid core of foam cells from the blood stream. The deep-ranging structural changes which are then present in the vessel wall are referred to in summary as plaque.

Arteriosclerotic plaque initially expands relatively little in the direction of the blood stream as the latter can expand as a compensation effect. With time however there is a constriction in the blood channel (stenosis), the first symptoms of which occur in physical stress. The constricted artery can then no longer expand sufficiently in order better to supply blood to the tissue to be supplied therewith. If it is a cardiac artery that is affected, the patient frequently complains about a feeling of pressure and tightness behind the sternum (angina pectoris). When other arteries are involved, painful cramps are a frequently occurring sign of the stenosis.

The stenosis can ultimately result in complete closure of the blood stream (cardiac infarction, stroke). Recent investigations have shown however that this occurs only in about 15 percent of cases solely due to plaque formation. Rather, the progressive breakdown of the fibrous cover layer of collagen, which is caused by certain inflammation mediators from the foam cells, seems to be a crucial additional factor. If the fibrous cover layer tears open the lipid core can come directly into contact with the blood. As, as a consequence of the inflammation reaction, tissue factors (TF) are produced at the same time in the foam cells, and these are very potent triggers of the coagulation cascade, the blood clot which forms can block off the blood vessel.

Non-operative stenosis treatment methods were established more than twenty years ago, in which inter alia the blood vessel is expanded again by balloon dilation (PTCA—percutaneous transluminal coronary angioplasty). It will be noted however that expansion of the blood vessel occasionally gives rise to injuries in the vessel wall, which admittedly heal without any problem but which in about a third of cases, due to the triggered cell growth, result in growths (proliferation) which ultimately result in renewed vessel constriction (restenosis). The expansion effect also does not eliminate the physiological causes of the stenosis, that is to say the changes in the vessel wall. A further cause of restenosis is the elasticity of the expanded blood vessel. After the balloon is removed the blood vessel contracts excessively so that the vessel cross-section is reduced (obstruction). The latter effect can only be avoided by the placement of a stent. The use of stents admittedly makes it possible to achieve an optimum vessel cross-section, but the use of stents also results in very minor damage which can induce proliferation and thus ultimately can trigger restenosis.

In the meantime extensive knowledge has been acquired in regard to the cell-biological mechanism and to the triggering factors of stenosis and restenosis. As already explained above restenosis occurs as a reaction on the part of the vessel wall to the expansion of the arteriosclerotic plaque. By way of complex active mechanisms lumen-directed migration and proliferation of the smooth muscle cells of the media and the adventitia is induced (neointimal hyperplasy). Under the influence of various growth factors the smooth muscle cells produce a cover layer of matrix proteins (elastin, collagen, proteoglycans) whose uncontrolled growth can gradually result in constriction of the lumen. Systematically medicinal therapy involvements provide inter alia for the oral administration of calcium antagonists, ACE-inhibitors, anti-coagulants, anti-aggregants, fish oils, anti-proliferative substances, anti-inflammatory substances and serotonin-antagonists, but hitherto significant reductions in the restenosis rates have not been achieved in that way.

The so-called concept of local drug delivery (LDD) provides that the active substance or substances is or are liberated directly at the location of the occurrence and limited to that area. For that purpose, a surface of the endovascular implant, that is to say in particular a stent, which faces towards the vessel wall, is generally provided with an active coating. The active component of the coating in the form of a therapeutic active substance can be bound directly to the surface of the implant or embedded in a suitable drug carrier. In the latter case the active substance is liberated by diffusion and possibly gradual breakdown of the biodegradable carrier.

Numerous preparations have been proposed as active substances and active substance combinations, but the effect which has been demonstrated hitherto in therapeutic tests is only moderate and the drugs used are in part highly cost-intensive.

PPAR-agonists have long been available as active substances for the treatment of type 2 diabetes and as lipid reducers. The term peroxisome proliferator activated receptors (PPAR) is used to embrace a class of steroid hormone-like nuclear receptors. At the present time three PPAR-isoforms, namely PPARα, PPARβ and PPARγ with subtypes γ₁ and γ₂ thereof are known at the present time. The term PPARβ is governed by historical considerations. That receptor was first found on the claw-toed frog. Later, PPARδ which in terms of development history comes from the β-receptor was found in higher animals. The receptor systems can be associated in functional respects with the steroid hormone receptors and are thus specific ligand-activated transcription factors, by means of which ligands (steroid hormones, peroxisome proliferators and many others) influence the synthesis of proteins if the corresponding gene is responsive. Known peroxisome proliferators which are foreign to the body are to be found among pharmaceutical active substances for the treatment of diabetes and hyperlipidemia and among insecticides, herbicides, fungicides, wood preservative agents, industrial lubricants and other xenobiotics.

Glitazones such as pioglitazone and rosiglitazone are increasingly used as anti-diabetic agents, in particular for the treatment of insulin resistance in relation to type 2 diabetes. The active substance group of glitazones have a thiazolidine-2,4-dione residue as a common functional group. It is assumed at the present time that the insulin sensitizers bind to nuclear PPARγ-receptors and binding causes the transcription of genes which are involved in adipocyte differentiation. Boosted expression of lipoprotein lipases, fatty acid transport enzymes and acyl-CoA-synthase was also observed, which cause a reduction in the triglyceride level and the free fatty acids. Together with further effects the oral application of glitazones results in an improvement in glucose utilization in muscle and fatty cells.

Lipid reducers have been used for some years for oral application as arteriosclerosis prophylaxis. As is known, endogenous triglycerides are encased in the liver in very low density lipoproteins (VLDL or pre-β-lipoproteins) and separated into the vascular stream. Certain lipoprotein-lipases cleave a part of the triglycerides out of the VLDL-particles, in which case the breakdown results in lipoproteins of lower density (LDL). The LDL-particles are the main cholesterol carriers of the plasma. The concentration thereof can rise on the one hand due to the increase in secretion and breakdown of the triglyceridic VLDL-particles and on the other hand due to reduced LDL-breakdown. LDL-breakdown occurs intracellularly predominantly for the synthesis of membranes, wherein firstly the LDL-particles are introduced into the cell by receptors arranged at the surface on the cell membrane. In the event of chronic surplus of LDL the number of LDL-receptors at the membrane surface falls so that LDL increasingly remains in the plasma and as already mentioned is deposited in the artery walls and is finally modified.

In the breakdown of VLDL or LDL respectively the surface substances which serve as dissolving intermediaries are partially given off. They can in turn encase fat and the resulting lipoprotein structures have a higher specific weight (HDL or α-lipoproteins). By virtue of their specific structure, the HDL-particles permit the binding of excess lipids (triglycerine or cholesterol) out of the tissues, that is to say also for example from the artery wall. High levels of HDL-concentration therefore permit cholesterol-loaded artery walls to be repaired. A good lipid-reducing therapy therefore aims at reducing VLDL and/or LDL or an increase in the level of HDL-concentration.

Inter alia use of the active substance clofibrate (2-(4-chlorophenoxy)-2-methylpropionic acid ethylester) is proposed for pharmacotherapy. Clofibrate, a PPARα-agonist, reduces the VLDL-synthesis in the liver and activates the lipoprotein-lipase. Subsequently the triglyceride level and to a lesser degree the cholesterol level of the plasmas is reduced and the level of HDL-concentration is immaterially increased. Further fibrates such as etafibrate, bezafibrate, fenofibrate and gemfibrozil are used in part as monomer-preparation and in part as a combination preparation in the same manner and sometimes achieve marked increases in the HDL-concentration.

Hitherto the above-mentioned PPAR-agonists have been used in practice exclusively for oral long-term application, in particular for the treatment of type 2 diabetes and for the prevention of arteriosclerosis. By virtue of the relatively high dose and the long-term use however undesired side-effects are to be likely to occur. Thus, studies in relation to long-term therapy with clofibrate reported on an increase in kidney and gall bladder diseases while in the case of PPARγ-agonists edema formation, increase in weight and hepatotoxicity were observed.

In accordance with more recent studies PPARs form heterodimers with a further form of nuclear receptors, the 9-cis-retinoic acid receptors (RXR). The PPAR/RXR-heterodimers bind to specific DNA-sequences which act as promoters of given genes, such as for example acyl-CoA-oxidase (AOX) or adipocytic fatty acid binding proteins (aP2). Binding of an agonist both to PPAR and also to RXR generally results in a change in the expression level of mRNA which is coded by the target genes of the heterodimer (transactivation).

The cytostatic bexarotene is used for therapy of cutaneous T-cell-lymphoma (CTCL). The precise active mechanism is not yet known. Presumably bexarotene as agonist binds to specific 9-cis-retinoic acid receptors. In vitro bexarotene inhibits the growth of degenerated hematopoetic cells. In vivo it prevents tumor regression or re-formation.

According to a study phytanic acid is said to be a natural rexinoid (RXR-agonist) (McCarty M. F.; The chlorophyll metabolite phytanic acid is a natural rexinoid—potential for treatment and prevention of diabetes; Medical Hypotheses 56 (2001) 217-219).

European Patent application 1 236 478, although published after the relevant date, discloses an active coating of a stent with at least one PPARγ-agonist. Glitazones are referred to as PPARγ-agonists.

The object of the present invention is not to intervene in the entire metabolism of the patient, but provide only locally therapeutic formulations for the treatment of stenosis or restenosis. The implants modified in accordance with the invention are intended to ensure improved compatibility, in particular in regard to any inflammatory and proliferative processes in the tissue environment.

### SUMMARY OF INVENTION

That object is attained by the endovascular implant, in particular a stent, with an at least portion-wise active coating, comprising the features recited in the appended claims. By virtue of the fact that the active coating as an active substance includes a or a combination of PPARγ-agonists and PPARδ-agonists or as the active substance an RXR-agonist or as the active substance a combination of PPAR-agonists and RXR-agonists, it is possible to effectively treat or prevent stenosis and also restenosis locally, that is to say only in the immediate environment of the implant. The only local application in very small amounts of active substance avoids side-effects as occur for example in the oral application of anti-diabetic agents and lipid reducers. Surprisingly it was found that neointima proliferation could be markedly reduced with active coatings of that kind. Evidently the local application of the above-mentioned PPAR-agonists or RXR-agonists in the region of damaged arterial vessel walls results in a marked reduction in inflammatory and proliferative processes.

The use of one or a combination of PPARα-agonists and PPARδ-agonists as an active substance for production of an active coating suitable for the local treatment of stenosis or restenosis on endovascular implants represents a new field of indication in respect of that group of active substances and is claimed in its full scope. The situation is just the same with a use of a combination of PPAR-agonists and RXR-agonists and a use of RXR-agonists as active substances for the production of an active coating for the local treatment of stenosis or restenosis. The active substance is prepared in a pharmacologically active form of application or as a pro-drug and a pharmacologically effective dosage on the surface of the implant.

Preferably, if the active substance includes PPARα-agonists, the active substance is a fibrate as that group of PPARα-agonists, in a situation involving local application, in the sense according to the invention, exhibit a positive therapeutic effect on restenosis and stenosis. In particular the fibrate is an active substance from the group of clofibrate, etofibrate, etofyllinclofibrate, bezafibrate, fenofibrate and gemfibrozil. It is precisely clofibrate that is distinguished by its ease of handling and working, its low price and its evidently anti-inflammatory and anti-proliferative effect on the tissue environment of the implant.

It is further preferred that, if the active substance includes a PPARγ-agonist, the active substance is a glitazone. The PPARγ-agonists, in particular ciglitazone, pioglitazone, rosiglitazone and troglitazone, evidently have an anti-inflammatory and anti-proliferative effect and thus reduce inter alia the risk of restenosis after implantation of a stent.

Bexarotene and phytanic acid are preferred as RXR-agonists.

In accordance with a preferred variant of the invention the PPAR/RXR-agonists are embedded in a drug carrier. That makes it possible to simplify the production of the coated implants and to control liberation of the drug. In addition, it is possible to effectively suppress unwanted flaking detachment of the active substance during the implantation procedure, in particular dilation of the stent. It will be appreciated that the drug carrier must be biocompatible. Preferably the drug carrier is additionally also biodegradable so that specific and targeted dosage of the drug is possible by way of a breakdown behavior on the part of the carrier. In this connection the use of polylactides, in particular poly-L-lactide and copolymers thereof (for example poly(L-lactide-co-trimethylene carbonate), poly(L-lactide-co-D/L-lactide)), polydioxanone and hyaluronic acid has proven to be particularly desirable.

A layer thickness of the active coating, in the case of drug carriers with an embedded active substance, is preferably between 5 and 30 μm, in particular between 8 and 15 μm. A mass by weight per implant, that is to say the weight of the drug carrier plus active substance, is preferably in the range of between 0.3 and 2 mg, in particular between 0.5 and 1.5 mg, particularly preferably between 0.5 and 1 mg. With the selected ranges, it is possible to achieve a high level of local action without the feared side-effects in kidneys, gall bladder and so forth occurring. Such thin coatings do not have a tendency to cracking and accordingly resist flaking detachment when a mechanical loading is applied (stent dilation).

If a biodegradable drug carrier is used then the elution characteristic can be influenced in particular by a variation in the degree of cross-linking of the polymer matrix or a variation in the degree of polymerization. Besides degradation of the carrier, diffusion processes are crucial in terms of elution of the active substance. Structural properties of the carrier (such as crystallinity, molecular weight, looping density) and of the active substance, besides many other factors, influence the diffusion rate. The elution characteristic of an active coating of that kind is preferably set in such a way that between 10 and 80%, in particular between 15 and 70%, particularly preferably between 15 and 25%, of the active substance is liberated within the first two days. The balance of the remaining active substance is to be successively delivered within the first months, also controlled by way of diffusion and degradation processes. It was surprisingly found that these periods which in themselves are relatively short already permit effective suppression of neointimal proliferation.

Preferably the fibrates are applied to the endovascular implant in a dose of between 0.05 and 1 mg, in particular between 0.1 and 0.75 mg, particularly preferably between 0.1 and 0.3 mg. The dose of glitazones per implant is preferably between 0.01 and 0.5 mg, in particular between 0.02 and 0.2 mg. The RXR-agonists bexarotene and phytanic acid are preferably used in dosages of between 5 and 100 μg, in particular between 10 and 100 μg. The dose of the active substances is so low that, even if the active substances are completely transported away by the blood plasma, as is assumed to occur, it is not necessary to reckon on a dose which stresses the organism overall. In contrast in local terms the dosage is sufficient to achieve the desired effect on restenosis prophylaxis.

As the diffusion processes take place starting from the implant surface into the intima and subsequently into the media of the vessel wall relatively slowly, the implant should be covered with the active coating over the largest possible surface area at its outside. Application of the active substance or of the active substance including a drug carrier is preferably effected with rotational atomizers which produce a finely distributed mist of very small suspended particles. The mist provides for surface wetting of very small structures on the implant and is then dried by being blown away. That procedure can be repeated as desired until the desired layer thickness is reached. If desired, it is possible in that way also to produce multi-layer systems—for example for the combination of various PPAR-agonists and RXR-agonists which are applied in succession. It is also possible in that way to produce a concentration gradient in the coating so that for example at the beginning of liberation an increased amount of the active substance can be eluted, which amount then successively decreases. It is also possible to envisage retardation of active substance liberation by an active substance-free polymer cover layer, referred to as a top coat.

It is further advantageous if a base body of the implant is formed from at least one metal or at least one metal alloy. It is further advantageous if the metal or the metal alloy is at least partially biodegradable. The biodegradable metal alloy can be in particular a magnesium alloy. The stent, in the biodegradable variant, is completely broken down with time and this means that possible triggers for an inflammatory and proliferative reaction of the surrounding tissue also disappear.

A stent design should preferably be so adapted that there is contact with the vessel wall over the largest possible surface area. That promotes uniform elution of the active substance which is substantially diffusion-controlled according to investigations. Regions of high mechanical deformability are preferably to be cut out in the coating as it is here that the risk of flaking detachment of the coating is increased. Alternatively or supplemental thereto the stent design can be so predetermined that, in the event of a mechanical loading, that is to say generally upon dilation of the stent, the forces occurring are distributed as uniformly as possible over the entire surface of the stent. It is possible in that way to avoid local overloading of the coating and thus crack formation or indeed flaking detachment of the coating.

The active coating has a very high level of adhesion capability if the implant has a passive coating of amorphous silicon carbide. The polymeric coating can be applied directly to the passive coating. Alternatively it is possible to provide spacers or bonding layers which are bonded to the passive coating for further enhancing the adhesion capability of the polymeric coating. Activation of the surface to be coated can also be envisaged, by means of plasma or by means of wet-chemical processes.

Further preferred configurations of the invention will be apparent from the other features which are set forth in the appendant claims.

### BRIEF DESCRIPTION OF THE DRAWINGS

The invention will be described in greater detail hereinafter by means of embodiments and with reference to the drawings in which:

FIG. 1 shows a diagrammatic plan view of a portion of an endovascular implant in the form of a stent,

FIG. 2 is a view in section through a structural element of the stent with an active coating, and

FIG. 3 shows a stent design which is an alternative to FIG. 1.

### DETAILED DESCRIPTION OF A PREFERRED EMBODIMENT

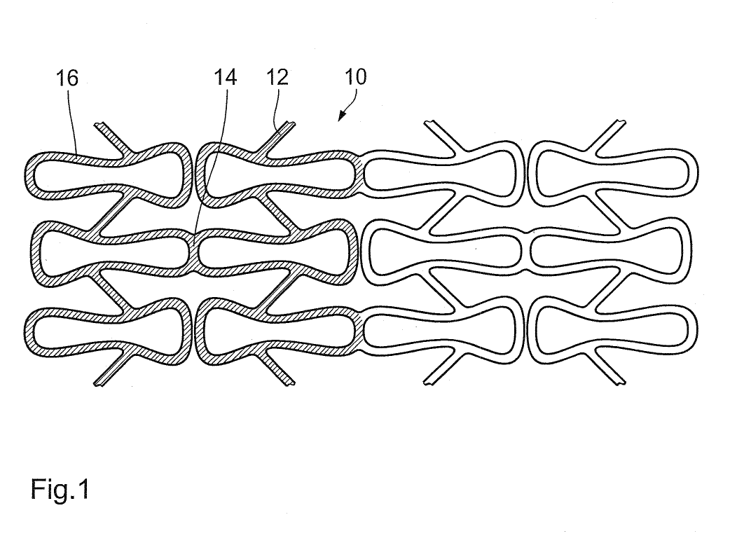

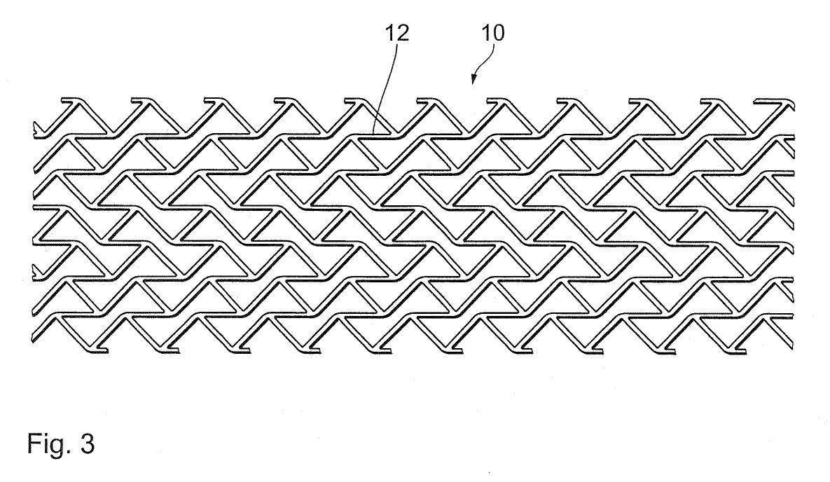

FIG. 1 is a diagrammatic view of a portion of an endovascular implant, here in the form of a stent 10. The stent 10 comprises a plurality of structural elements 12 which—as illustrated in this specific example—form a lattice-like pattern about the longitudinal axis of the stent 10. Stents of this kind have long been known in medical technology and, as regards their structural configuration, can vary to a high degree. What is of significance in regard to the present invention is that the stent 10 has an outwardly facing surface 14, that is to say a surface which is directed towards the vessel wall after implantation. In the expanded condition of the stent 10 that outward surface 14 should involve an area coverage which is as large as possible in order to permit uniform active substance delivery. In regard to the mechanical basic structure, distinctions are to be drawn in terms of the configuration involved: concentration of the deformation to a few regions or uniform deformation over the entire basic structure. In the former case, the structures are such that, upon mechanical expansion of the stent, there are only deformations concentrated in the region of flow hinges (thus for example in the stent 10 shown in FIG. 1). The second variant in which dilation results in deformation of virtually all structural elements 12 is shown by way of example in FIG. 3. It will be appreciated that the invention is not limited to the stent patterns illustrated. Modifications in the stent design which increase the contact surface area are generally preferred as, in the case of active substance-laden coatings, that permits more uniform elution into the vessel wall. In addition, regions involving a high level of mechanical loading, such as for example the flow hinges in FIG. 1 are either not to be coated or a stent design is predetermined (for example that shown in FIG. 3), which distributes the forces occurring upon dilation to all structures of the stent more uniformly. That is intended to avoid crack formation or flaking detachment of the coating as a consequence of the mechanical loading.

The surface 14 of the structural elements 12 is covered with an active coating 16, indicated here by a surface with dark hatching. The active coating 16 extends over the entire surface 14 or—as shown here—only over a portion of the surface 14. The active coating 16 comprises one or a combination of PPAR-agonists and/or RXR-agonists which were applied in their pharmacologically active form of application to the surface 14 of the structural elements 12 and adhere thereto.

The term “pharmacologically active form of application” is used to denote all properties of the active substance in terms of morphology and solubility of the substance or the salts thereof, which contribute to ensuring reproducible dosage in accordance with a therapeutic treatment. Thus it is frequently advantageous to use amorphous microcrystalline active substance modifications as they exhibit a particularly rapid and uniform elution behavior.

As active substance, the active coating 16 contains fibrates, glitazones, bexarotene and/or phytanic acid. The substances involved are in particular fibrates from the group of clofibrate, etofibrate, etofyllinclofibrate, bezafibrate, fenofibrate and gemfibrozil and glitazones from the group of ciglitazone, pioglitazone, rosiglitazone and troglitazone. It has now surprisingly been found that these active substances can also be successively used in local application for the prevention of restenosis.

The active coating 16 may also include a drug carrier which is biocompatible and permits controlled liberation of the active substance. In addition the drug carrier also serves for improved bonding of the active coating 16 to the stent surface 14 in order to prevent flaking detachment of the active coating 16 upon dilation or introduction of the stent 10 into an arterial vessel. The drug carriers which are distinguished in this respect are in particular hyaluronic acid and polylactides including their copolymers such as for example poly(L-lactide-co-trimethylene carbonate) or poly(L-lactide-co-D,L-lactide) and also polydioxanone.

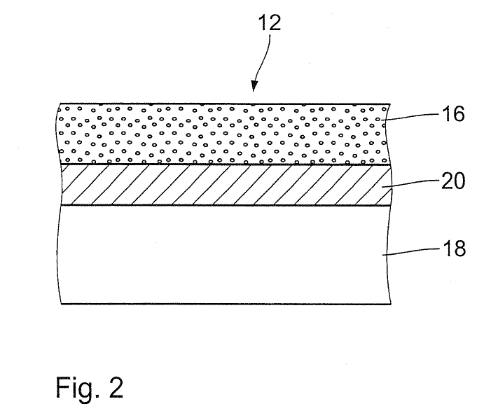

A particularly high degree of adhesion to the surface of the structural elements 12 can be achieved if the stent 10 at its surface 14 additionally has a passive coating 20 of amorphous silicon carbide (see FIG. 2). The production of structures of that kind is known from the state of the art, in particular from patent DE 44 29380 C1 to the present applicants, to the disclosure of which attention is directed in respect of the full extent thereof, and it is therefore not to be described in greater detail at this point. It merely remains to be emphasized that the adhesion capability of the active coating material to the stent surface 14 can be improved with such a passive coating 20. In addition the passive coating 20 on its own already reduces neointimal proliferation.

A further improvement in the adhesion capability can be achieved if bonding of the polymeric carrier material is effected covalently by means of suitable spacers or by applying a bonding layer (not shown here). The essential traits of activation of the silicon carbide surface are to be found in published German application DE 195 33 682 A1 to the present applicants, to the disclosure of which attention is hereby directed in respect of the full extent thereof. The spacers used can be photoreactive substances such as benzophenone derivatives which, after reductive coupling to the substrate surface and possibly protection removal, provide functional binding sites for the polymer. A bonding layer which is a few nanometers thick can be achieved for example by silanization with epoxyalkylalkoxy silanes or epoxyalkylhalogen silanes and derivatives thereof. The polymeric carrier material is then bound to the bonding layer by physisorption or chemisorption. The procedure is suitable in particular for polymeric carrier materials polylactide and hyaluronic acid.

FIG. 2 is a view in section through a structural element 12 of the stent 10 in any region thereof. The active coating 16 is applied to a base body 18 with the above-mentioned passive coating 20 of amorphous silicon carbide. The base body 18 can be formed from metal or a metal alloy. If the entire stent 10 is to be biodegradable the base body 18 can be produced in particular on the basis of a biodegradable metal or a biodegradable metal alloy. A biodegradable magnesium alloy is particularly suitable. Materials of that kind are also already adequately described in the state of the art so that they will not be especially set forth here. In this connection attention is directed in particular to the disclosure in DE 198 56983 A1 to the present applicants.

If the drug carrier is biodegradable the elution characteristic of the active substance can be influenced by varying the degree of cross-linking of the polymer matrix or a variation in the degree of polymerization. The procedure is suitable in particular for the drug carriers hyaluronic acid or polylactide. With an increasing degree of cross-linking and an increasing molecular mass of the polymer, the period of time over which the active substance is liberated is generally also increased. The elution characteristic of an active coating of that kind is preferably set in such a way that between 10 and 80%, in particular between 15 and 70%, particularly preferably between 15 and 25%, of the active substance is liberated within the first two days. The balance of the remaining active substance is to be delivered successively within the first months, also controlled by way of diffusion and degradation processes.

The active coating 16 can also be structured in its makeup. For example a lower degree of cross-linking can be provided in the outer regions of the active coating 16, than in the further inwardly disposed regions. In that way, breakdown of the active coating 16 after implantation can initially take place more rapidly and, with a uniform level of active substance concentration in the active coating 16, an overall higher initial dose can be liberated, than in the remaining period of time. Alternatively or in addition, that effect can be achieved by predetermining locally different levels of concentration of the active substance in the active coating 16, for example by the uppermost regions of the coating 16 having higher concentrations of active substance.

Production of the active coating 16 is implemented by means of a rotational atomizer which produces a mist of micro-fine particles. Alternatively it is also possible to use ultrasonic atomizers. The coating operation is effected stepwise in numerous cycles which comprise a step of wetting the stent in the spray mist produced and a subsequent step of drying the deposit on the stent by blowing it away. The multi-stage production process makes it possible to produce any layer thicknesses and—if desired—concentration gradients of the active substance or substances in individual layers of the active coating 16. Sterilization of the stent is effected by electron bombardment, in which case partial cracking of the polymer chains of a polymeric carrier that is possibly provided, with high molecular weights of the polymer, can be tolerated. The kinetic energy of the electrons is approximately in the range of between 3.5 and 6 Mev, in particular between 4 and 5 MeV as, at those values, adequate sterilization with an only slight degree of depth of penetration is still ensured. The dosage ranges between 15 and 45 kGy, in particular between 15 and 35 kGy per stent. Investigations showed that no or only a minimal reduction in the biological activity of the active substances occurs due to the sterilization process.

The layer thicknesses produced for the active coating 16 are generally in the range of between 5 and 30 μm. Layer thicknesses in the range of between 8 and 15 μm are particularly desirable as that already ensures very substantial coverage of the surface 14 of the stent 10 and it is not yet necessary to reckon on the occurrence of structural problems such as crack formation and the like. Overall between about 0.3 and 2 mg, in particular between 0.5 and 1.5 mg, of coating material is applied per endovascular implant, if the active coating 16 includes a drug carrier. A dose of the active substance when using fibrates is in the range of between 0.05 and 1 mg, in particular between 0.1 and 0.75 mg, while when using glitazones it is in the range of between 0.01 and 0.5 mg, in particular between 0.02 and 0.2 mg. Bexarotene and phytanic acid are applied with a dose in the range of between 5 and 100 μg.

### Embodiment

A commercially available stent which can be obtained under the trade name LEKTON from BIOTRONIK is used in the endovascular implant.

The stent is clamped in a rotational atomizer. A solution of poly-L-lactide (which can be obtained under the trade name RESOMER L214 from Boehringer Ingelheim) and clofibrate in chloroform is prepared in a supply container of the atomizer (poly-L-lactide concentration: 7.5 g/l). The proportion by weight of the active substance clofibrate to the mass of the drug carrier poly-L-lactide is set to between about 10% and 50%, in particular between 15% and 40%, preferably between 20% and 30%, of the total mass. Active substance concentrations of 15%, 30% and 40% were tried.

The stent is wetted on one side with a finely distributed mist produced by the rotational atomizer in 80 cycles each of a duration of about 10 s. The respective wetting operation is followed by a drying step by blowing-off of a duration of about 12 seconds. After termination of the single-sided coating procedure the rear side of the stent is coated in accordance with the procedure just described above.

After the end of a total of 160 coating cycles the stent is removed and sterilized by electron bombardment. The layer thickness of the active coating is about 10 μm and the mass of the active coating is about 0.7 mg, giving an active substance mass of about 140 μg per stent.

The stent was tested in animal experiments on the cardiovascular system of a pig. For that purpose the stent was alternately implanted in the Ramus interventricularis anterior (RIVA), Ramus circumflexus (RCX) and the right coronary artery (RCA) of the heart of 7 pigs. For comparative purposes at the same time a blind test was started with stents without a coating. After 4 weeks the restenosis rates of the stents with and without active coating were determined by measuring off the level of neointimal proliferation by means of quantitative coronary angiography and compared. There was a significant reduction in neointimal proliferation when using a stent with an active coating.

What is claimed is:

1. An endovascular implant, comprising:
an active coating comprising a combination of PPARδ-agonists and RXR-agonists as an active substance on at least a portion of the endovascular implant.

2. The implant of claim 1, wherein:
the active substance further comprises a fibrate from the group consisting of clofibrate, etofibrate, etofyllinclofibrate, bezafibrate, fenofibrate and gemfibrozil.

3. The implant of claim 2, wherein:
the active coating further comprises a drug carrier from the group consisting of polylactide, poly-L-lactide and hyaluronic acid.

4. The implant of claim 1, wherein:
the active substance further comprises a glitazone from the group consisting of ciglitazone, pioglitazone, rosiglitazone and troglitazone.

5 The implant of claim 4, wherein:
the active coating further comprises a drug carrier from the group consisting of polylactide, poly-L-lactide and hyaluronic acid.

6. The implant of claim 1, wherein:
the active substance further comprises bexarotene or phytanic acid.

7. The implant of claim 6, wherein:
the active coating further comprises a drug carrier from the group consisting of polylactide, poly-L-lactide and hyaluronic acid.

8. The implant of claim 1, wherein:
the active coating further comprises a drug carrier from the group consisting of polylactide, poly-L-lactide and hyaluronic acid.

9. An endovascular implant, comprising:
an active coating comprising a PPARδ-agonist as an active substance on at least a portion of the endovascular implant.

10. The implant of claim 9, wherein:
the active substance further comprises a fibrate from the group consisting of clofibrate, etofibrate, etofyllinclofibrate, bezafibrate, fenofibrate and gemfibrozil.

11. The implant of claim 10, wherein:
the active coating further comprises a drug carrier from the group consisting of polylactide, poly-L-lactide and hyaluronic acid.

12. The implant of claim 9, wherein:
the active substance further comprises a glitazone from the group consisting of ciglitazone, pioglitazone, rosiglitazone and troglitazone.

13. The implant of claim 12, wherein:
the active coating further comprises a drug carrier from the group consisting of polylactide, poly-L-lactide and hyaluronic acid.

14. The implant of claim 9, wherein:
the active substance further comprises bexarotene or phytanic acid.

15. The implant of claim 14, wherein:
the active coating further comprises a drug carrier from the group consisting of polylactide, poly-L-lactide and hyaluronic acid.

16. The implant of claim 9, wherein:
the active coating further comprises a drug carrier from the group consisting of polylactide, poly-L-lactide and hyaluronic acid.

17. An endovascular implant, comprising:
an active coating comprising an RXR-agonist as an active substance on at least a portion of the endovascular implant.

18. The implant of claim 17, wherein:
the active substance further comprises bexarotene or phytanic acid.

19. The implant of claim 18, wherein:
the active coating further comprises a drug carrier from the group consisting of polylactide, poly-L-lactide and hyaluronic acid.

20. The implant of claim 17, wherein:
the active coating further a drug carrier from the group consisting of polylactide, poly-L-lactide and hyaluronic acid.
